(19) United States
(12) Patent Application Publication
(10) Pub. No.: US 2026/0306756 A1
(43) Pub. Date: Oct. 8, 2026

(54) Nebulisation Method

(71) Applicant: ACU-FLOW LIMITED, Glasgow (GB)

(72) Inventors: Christian Witte, Glasgow (GB); Elijah Nazarzadeh, Glasgow (GB); John Pritchard, Glasgow (GB)

(21) Appl. No.: 19/478,807

(22) PCT Filed: Apr. 25, 2024
(86) PCT No.: PCT/EP2024/061412

§ 371 Date: Oct. 25, 2025

(30) Foreign Application Priority Data
Apr. 25, 2023 (GB) 2306063.5

(51) Int. Cl. A61M 11/00

(52) U.S. Cl. CPC A61M 11/005; A61M 11/001

(57) ABSTRACT

A method is disclosed for the preparation of nebulised droplets. A device is provided having an acoustic wave interaction surface with an array of cavities opening to the acoustic wave interaction surface. An acoustic wave transducer generates and propagates acoustic waves to the acoustic wave interaction surface. A liquid for nebulisation is held in the cavities. The acoustic wave transducer is operated at a first input power to produce nebulised droplets having a first aerosol particle size distribution. The acoustic wave transducer is operated at a second input power, greater than the first input power, to produce nebulised droplets having a second aerosol particle size distribution having a larger mass median aerodynamic diameter (MMAD) than that of the first aerosol particle size distribution.

### FIELD OF THE INVENTION

The present invention relates to methods and apparatus for nebulisation of liquids including liquid suspensions. Such methods and apparatus are of particular, although not necessarily exclusive, interest for application in the delivery of therapeutic agents to subjects.

### BACKGROUND

According to the World Health Organization (WHO) there are 262 million people suffering from asthma and 392 million people with chronic obstructive pulmonary disease (COPD), leading to 3.7 million deaths per year worldwide. Many millions also suffer with pulmonary infectious disease, cystic fibrosis, pulmonary hypertension and allergic rhinitis as well as other under-diagnosed chronic respiratory diseases. Estimates of the cost of treating patients with such lung diseases, including those caused by tuberculosis (TB), COPD, cystic fibrosis, pneumonia, asthma and smoking was Euro 380 billion per annum (according to the European Lung White Book of the European Respiratory Society http://www.erswhitebook.org/[accessed 7 Oct. 2022]).

Inhalation drug delivery is known as a particularly suitable route for treatment of respiratory disorders. One of the major advantages of the inhalation pulmonary route is that it can be targeted directly to the lung, and indeed, effective delivery of medication has been shown to be crucially dependent upon the droplet size distribution within the aerosol of medicine (according to Brun et al (2000)). In general, if the size is too small (<0.5 μm), the droplet will be exhaled, whilst if the size is too large (>9.0 μm), the droplet will be trapped in the upper respiratory tract or throat. It is considered that pulmonary drug delivery requires droplet size distribution of the aerosols with diameters between about 1 and 5 μm. To treat the nasal passages, droplets up to 100 μm may be required.

When aerosol particle size is fixed it cannot be tailored to provide optimised delivery of different drugs for different diseases. This leads to sub-optimal performance (for example, corresponding to <30% drug delivered) which is particularly unsuitable for drugs which are expensive and/or have low potency. As a result, the use of current nebuliser technology is restricted to very young or old patients suffering from diseases such as cystic fibrosis or obstructive pulmonary disorder or for those with very poor lung function, where a high payload of drug is required. A further issue is that certain drug formulations cause nebuliser devices with an unsuitable APSD to malfunction, affecting the quality of the drug formulations. For example, water-insoluble active pharmaceutical ingredients (API) can be formulated as suspended particles in liquid, known as suspension formulations. The suspension formulations are found to be challenging for nebulisation, especially for nebulisation with ultrasonic nebulisers. Ultrasonic nebulisers use a focused beam of ultrasonic waves to cause droplets to detach from the liquid to create an aerosol. When ultrasonic nebulisers are operated at high power intensities, the suspended particles aggregate and cannot be dispersed with the aerosol.

Nebulisation of suspensions using mesh nebulizers is limited by the size of the mesh. For instance, marketed budesonide (Pulmicort™) suspensions have a mean particle size in the suspension of 3-4 μm, whilst the mesh orifice size in mesh nebulisers is usually 2-3 μm. Thus, the budesonide particles usually block the orifices of the mesh preventing a significant portion of the drug suspension being delivered.

Further issues are encountered by surface active drugs which can flood the device, and biologics which can be subject to deleterious shear stress. Accordingly, some drugs cannot be delivered using nebulisation while other drugs require a specific nebuliser device for delivery to the user. These technical limitations translate to poor user experience typically associated with cleaning, portability, treatment time and high noise, which decreases user adherence to treatment.

The ability to control aerosol particle size distribution (APSD) on demand is a lacking feature in currently available nebuliser products. This capability could facilitate targeted drug delivery to the lungs and enhance treatment for various lung diseases and represents part of the insights of the present inventors on which the present disclosure is based. Additionally, delivery of drugs to the throat and nasal passages (for example) could be controlled using larger aerosol particles. Recent advancements in nebuliser technology, as set out in Nazarzadeh et al (2017 & 2019) and Pritchard et al (2022), have demonstrated that APSD can be controlled to an extent through physical confinement in microstructures that regulate capillary waves at the air-liquid interface. However, during operation, it may be impractical to alternate between nebulisers with different microstructures in order to control the APSD on demand. Therefore, it is desirable to provide a nebuliser device with a tuneable APSD capability.

The present invention has been devised in light of the above considerations.

### SUMMARY OF THE INVENTION

The present invention is based on the inventors' findings that the aerosol particle size distribution (APSD) can be tuned by controlling the input power applied to the nebuliser device and/or altering the flow rate of liquid for nebulisation, in the context of a nebuliser device having a particular type of construction and operation. Using this approach, the inventors have shown that the aerosol droplet size can be tuned, without the need to change the hardware. As described herein, the present invention is particularly suited to tuning the median aerosol droplet size between 0.7 μm and 5 μm, providing a single platform for targeted delivery to different regions of the lung or for the delivery of different medicaments.

In a first aspect, the present invention provides a method for the preparation of nebulised droplets, the method including the steps:
providing a device having an acoustic wave interaction surface with an array of cavities opening to the acoustic wave interaction surface;
providing an acoustic wave transducer adapted to generate and propagate acoustic waves to the acoustic wave interaction surface;
providing, in the cavities, a liquid for nebulisation;
operating the acoustic wave transducer at a first input power to generate and propagate acoustic waves to the acoustic wave interaction surface to interact with the liquid in the cavities to produce nebulised droplets having a first aerosol particle size distribution; and
operating the acoustic wave transducer at a second input power, greater than the first input power, to generate and propagate acoustic waves to the acoustic wave interaction surface to interact with the liquid in the cavities to produce nebulised droplets having a second aerosol particle size distribution having a larger mass median aerodynamic diameter (MMAD) than that of the first aerosol particle size distribution.

Without wishing to be bound by theory, the present inventors consider that the effect of locating the liquid in the cavities is that the liquid is pinned by the cavities, suppressing the formation of large droplets by capillary waves. In the context of the present invention, capillary waves can be considered to be waves which are capable of travelling along the free surface of the liquid, whose dynamics are dominated by surface tension effects. In common terminology, they can be considered to be “ripples” in the manner of ripples on the surface of a body of water.

During nebulisation, the acoustic waves overcome the surface tension of the liquid at the acoustic wave interaction surface to form a thin liquid film. Without wishing to be limited by theory, the present inventors believe that acoustic energy carried by the acoustic waves influences the wetting behaviour of the array of cavities, the thickness of the liquid film, and the amplitude of the capillary waves. As such, acoustic wetting and capillary effects facilitate the spreading of thin liquid films to the array of cavities.

However, as the input power increases, more energy is absorbed into the liquid, leading to an increase in both capillary wave amplitude and the impact of acoustic streaming flows that counteract the spreading of the thin liquid film (Collins et al (2012)). Decreased liquid film spreading leads to enhanced interaction between the acoustic waves and the bulk liquid generating an acoustic body force, which results in the formation of larger aerosol droplets.

In a second aspect, the present invention provides a method for the preparation of nebulised droplets, the method including the steps:
providing a device having an acoustic wave interaction surface with an array of cavities opening to the acoustic wave interaction surface;
providing an acoustic wave transducer adapted to generate and propagate acoustic waves to the acoustic wave interaction surface;
providing, in the cavities, a liquid for nebulisation by metering the liquid for nebulisation to the cavities at a flow rate;
operating the acoustic wave transducer to generate and propagate acoustic waves to the acoustic wave interaction surface to interact with the liquid in the cavities to produce nebulised droplets having an aerosol particle size distribution; and
altering the flow rate of liquid for nebulisation to the cavities and thereby altering the aerosol particle size distribution of the nebulised droplets.

It is intended that altering the flow rate of liquid for nebulisation consists of altering the flow rate between different non-zero values.

The first and second aspects of the invention may be combined. For example, the method according to the first aspect may include providing the liquid for nebulisation in the cavities by metering the liquid for nebulisation to the cavities at a flow rate and altering said flow rate, thereby altering the aerosol particle size distribution of the nebulised droplets.

The method may include varying the input power and the flow rate simultaneously, e.g. while maintaining a constant ratio of the input power to the flow rate. For example, the method may include increasing the input power and the flow rate simultaneously, or decreasing the input power and the flow rate simultaneously, thereby maintaining a constant aerosol particle size distribution of the nebulised droplets. More generally, varying the input power and the flow rate together may assist in providing control over the time sequence of aerosol particle size distribution of the nebulised droplets.

The first aerosol particle size distribution of the first aspect may correspond to the first aerosol particle size distribution of the second aspect. When both steps of increasing the input power and the altering the flow rate are performed, the second aerosol particle size distribution of the first aspect may correspond to the second aerosol particle size distribution of the second aspect. This may provide an additional effect of increasing the mass per unit time of generated aerosol.

Furthermore the first and second aspects of the invention may have any one of or, to the extent that they are compatible, any combination of the following optional features.

Each cavity may be open at an end opposite to the acoustic wave interaction surface in fluid communication with a reservoir of the liquid to be drawn up by capillarity into the cavities to replace liquid lost by nebulisation. Preferably, the level of the liquid in the cavities is below the acoustic wave interaction surface during nebulisation. The level of the liquid in the cavities may be controlled by increasing or decreasing the flow rate. Additionally, or alternatively, the level of liquid in the cavities may be controlled by increasing or decreasing the input power.

The transducer preferably comprises at least one arrangement of electrodes. More preferably, the transducer comprises two or more arrangements of electrodes. For example, the electrodes may be interdigitated. The input power may be controlled by manipulating a drive signal from a signal generator, e.g. via the at least one arrangement of electrodes. An amplifier may be used to increase the voltage of the drive signal received by the transducer. Increasing or decreasing the input power may be performed by increasing or decreasing the amplifier gain, respectively. Alternatively, or additionally, increasing or decreasing the input power may be performed by increasing or decreasing the amplitude or peak-to-peak voltage of the drive signal from the signal generator.

The method may include adjusting the input power to the acoustic wave transducer from the first input power to the second input power and/or adjusting the input power of the acoustic wave transducer from the second input power to the first input power.

In the first aspect, the method may include operating the acoustic wave transducer at a third input power, greater than the second input power, to generate and propagate acoustic waves to the acoustic wave interaction surface to interact with the liquid in the cavities to produce nebulised droplets having a third aerosol particle size distribution having a larger mass median aerodynamic diameter (MMAD) than that of the second aerosol particle size distribution.

The method may include adjusting the input power to the acoustic wave transducer from the second input power to the third input power and/or adjusting the input power to the acoustic wave transducer from the third input power to the second input power.

The input power to the acoustic wave transducer may be continuously or stepwise variable. The input power may be varied through a range of values not less than 100 mW, not less than 200 mW, not less than 500 mW, not less than 1 W, not less than 2 W, or not less than 4 W. The input power may be varied through a range of values not more than 100 W, not more than 50 W, not more than 20 W, or not more than 10 W.

The method may include a power calibration step of calibrating the input power based on the first aerosol particle size distribution. The power calibration step may include calculating an average particle size of the nebulised droplets from the first aerosol particle size distribution and varying the input power based on the first average particle size.

The power calibration step may include determining a desired average particle size and comparing the average particle size to the desired average particle size. For example, the power calibration step may include increasing the input power if the average particle size is less than the desired average particle size and/or decreasing the input power if the average particle size is more than the desired average particle size.

Altering the flow rate may include operating the device using a first flow rate to produce nebulised droplets having a first aerosol particle size distribution and operating the device using a second flow rate, smaller than the first flow rate, to produce nebulised droplets having a second aerosol particle size distribution having a larger mass median aerodynamic diameter (MMAD) than that of the first aerosol particle size distribution.

The method may include altering the flow rate from the first flow rate to the second flow rate and/or altering the flow rate from the second flow rate to the first flow rate.

In the second aspect, altering the flow rate may include operating the device using a third flow rate, smaller than the second flow rate, to produce nebulised droplets having a third aerosol particle size distribution having a larger mass median aerodynamic diameter (MMAD) than that of the second aerosol particle size distribution.

The method may include altering the flow rate from the second flow rate to the third flow rate and/or altering the flow rate from the third flow rate to the second flow rate.

The method may include a flow rate calibration step of calibrating the flow rate based on the first aerosol particle size distribution. The flow rate calibration step may include calculating an average particle size of the nebulised droplets from the first aerosol particle size distribution and altering the flow rate based on the average particle size.

The flow rate calibration step may include determining a desired average particle size and comparing the average particle size to the desired average particle size. For example, the flow rate calibration step may include increasing the flow rate if the average particle size is more than the desired average particle size and/or decreasing the input power if the average particle size is less than the desired average particle size.

Operation of the device may result in a nebulised plume of droplets of average diameter in the range 1 to 100 μm. For example, the nebulised plume of droplets may have an average diameter in the range 1 to 80 μm, e.g. 1 to 50 μm, e.g. 1 to 30 μm, e.g. 1 to 20 μm, e.g. 1 to 10 μm, e.g. 1 to 5 μm, e.g. 1 to 3 μm, or in the range 2 to 100 μm, e.g. 4 to 100 μm, e.g. 6 to 100 μm, e.g. 8 to 100 μm.

The liquid may be one or more of: a pure compound; a mixture of liquids; a solution of one or more solutes in a liquid solvent; a suspension of particles (solid, substantially solid or liquid) in a carrier liquid; a colloid; an emulsion; nanoparticles or a suspension of nanoparticles.

The liquid may comprise an active pharmaceutical ingredient.

The acoustic wave interaction surface may be a surface acoustic wave (SAW) interaction surface and the acoustic wave transducer may be a SAW transducer adapted to generate and propagate SAWs to the SAW interaction surface. The present invention is not necessarily limited to SAWs and other acoustic waves can be used. Accordingly, the acoustic wave transducer may be operable to generate and/or propagate acoustic modes selected from one or more of Rayleigh waves (i.e. SAWs), Lamb waves, bulk acoustic waves and thickness mode.

The acoustic wave interaction surface may be a surface of the acoustic wave transducer. However, more preferably, the acoustic wave interaction surface is a surface of a superstrate coupled to the acoustic wave transducer.

The present invention is not necessarily limited to any particular orientation. The term “superstrate” is used because in typical implementations of embodiments of the invention, this item is placed on top of the acoustic wave transducer. However, other orientations are contemplated, e.g. in which a corresponding substrate is placed under the transducer and liquid is nebulized from the surface of the substrate.

The array of cavities may be formed in a substrate that is also the substrate for the acoustic wave transducer. Alternatively, the array of cavities can be formed in a separate superstrate/substrate that is configured to be acoustically coupled to the acoustic wave transducer.

Furthermore, the present invention is not necessarily limited to a planar configuration. For example, the transducer may be formed inside the superstrate, e.g. in a tubular configuration. Alternatively, the transducer may be formed around the superstrate, with the superstrate in the form of a tube (or hollow needle) held inside a transducer tube. This may be preferred, in order that a continuous (or quasi continuous) supply of sample fluid may be provided to the superstrate tube, with the nebulized plume provided at a free end of the superstrate tube.

Preferably, the superstrate is formed of a material which is impervious to the liquid. This helps to avoid any (potentially contaminating) contact between the transducer and the liquid.

The superstrate may be permanently coupled to the piezoelectric layer, in the sense that it is not removable from the piezoelectric layer without damage to the device.

Alternatively, coupling between the transducer and the superstrate may be achieved using a coupling medium, preferably a fluid or gel coupling medium. The coupling medium may be an aqueous coupling medium, e.g. water. Alternatively, the coupling medium may be an organic coupling medium, such as an oil-based coupling medium or glycerol. The coupling medium provides intimate contact between the superstrate and the transducer and allows the efficient transfer of acoustic energy to the superstrate from the transducer. The coupling medium may consist of or comprise the liquid to be subjected to nebulisation.

The advantage of providing the superstrate as a separate entity from the transducer is very significant. Acoustic wave transducers, and particularly SAW transducers, are complex to manufacture and therefore typically expensive. Contamination of the transducer may be difficult or impossible to remove if the liquid is allowed to come into contact with the transducer. Alternatively, removal may not be cost-effective, or may damage the transducer. However, it is strongly preferred that the transducer can be re-used. Accordingly, it is preferred that the liquid does not contact the transducer but instead contacts the superstrate coupled to the transducer. The superstrate itself may be disposable (e.g. disposed of after a single use). The superstrate may be formed by various methods, such as microfabrication, embossing, moulding, spraying, lithographic techniques (e.g. photolithography), etc.

The cavities preferably have substantially the same shape. The acoustic wave interaction surface, in use, preferably is held substantially horizontal. In this way, the cavities preferably open in the upward direction. The cavities may be closed at an end distal from the acoustic wave interaction surface. Alternatively, the cavities may be open at an end distal from the acoustic wave interaction surface.

The cavities may be substantially columnar in shape. In this way, the cross sectional shape of the cavities may be substantially uniform with depth (a direction perpendicular to the acoustic wave interaction surface). For example, the cross sectional shape of the cavities in the depth direction may be rectangular, square, rounded, oval, elliptical, circular, triangular, star-shaped. Most preferably the cross sectional shape of the cavities in the depth direction is circular. The cross sectional area of the cavities may be uniform with depth. However, in some embodiments this may not be the case, allowing the cavities to have a cross sectional area which narrows, expands or undulates with depth. For example, funnel-shaped cavities may be provided (such cavities being capable of being formed using a KOH etch for example), to provide suitable volume in the cavity to retain the liquid.

The cavities may have an internal structure. For example, there may be provided one or more pillars upstanding in the cavities, walls projecting into the cavities or other projections into the cavities. The internal walls of the cavities may have one or more array of such projections. The array of projections may be considered to be a phononic structure, in the sense that it is based on a periodic arrangement (in the manner disclosed in WO 2011023949, WO 2011060369, WO 2012114076 and WO 2012156755) for affecting the distribution and/or transmission of acoustic waves in the cavities. In the case of one or more pillars, there may be provided one or more support struts extending to the pillar to hold it in position. This is particularly the case if the cavity has two open ends (i.e. extends through the superstrate) since in this case there is no base of the cavity for the pillar to be supported on.

Such internal structure interacts with the liquid and with the acoustic waves in a manner which can further improve the performance of the cavities in controlling the APSD.

The cavities preferably have substantially the same dimensions.

Preferably the depth of the cavities is at least 500 nm, at least 800 nm, at least 1 μm, at least 2 μm, at least 5 μm, at least 10 μm, at least 20 μm, at least 50 μm, at least 100 μm, at least 200 μm, at least 400 μm, at least 500 μm, or at least 600 μm. In some embodiments, the cavities can be blind cavities. However, in other embodiments the cavities can open at a surface opposite to the SAW transmission surface. This is preferred, for example, where the liquid to be nebulised is fed to the cavity from one or more reservoirs.

Preferably the maximum dimension of the cavities in a direction perpendicular to the depth of the cavities is at least 100 μm. This lower limit is set in view of the preferred lower limit for droplet size. The lower limit may be at least 200 μm, at least 500 μm, at least 1 mm, at least 1.5 mm, or at least 2 mm. Where the cavities have a circular cross section shape, this dimension is referred to as the diameter D of the cavities. Where the cavities have a non-circular cross sectional shape, this maximum dimension is also referred to as the diameter. In some embodiments, the maximum dimension of the cavities in a direction perpendicular to the depth of the cavities may be at most 4 mm, at most 3 mm, at most 2 mm, or at most 1 mm.

The cavities can be in the form of cylindrical holes. As indicated above, in some embodiments the holes can be blind holes. In other embodiments, the holes can be holes which open also at an opposing surface to the acoustic wave interaction surface, in order that additional liquid can be fed into the cavities by capillarity. A suitable volume for the cavities in either can be at least 0.5 nl, more preferably at least 1 nl. This volume is preferably at most 50 nl, more preferably at most 20 nl, more preferably at most 10 nl, more preferably at most 5 nl. As an example, a cylindrical hole of diameter 100 μm and depth 300 μm has a volume of about 2 nl.

The array of cavities may not have long range order. In this case, the arrangement of the cavities may be substantially random, in the sense of not being based on a periodic arrangement.

It is preferred that the cavities have an average cavity-to-cavity nearest neighbour spacing (measured from the central axis of each cavity) of at least 200 μm, although as will be understood the lower limit for this will typically be the diameter of the cavities. As a guide, a cavity diameter of about 10 μm would be suitable for acoustic waves in the MHz region (e.g. of frequency of around 100 MHz). More preferably, this spacing is at least 250 μm, at least 300 μm, at least 400 μm, at least 500 μm, at least 600 μm, at least 700 μm, at least 800 μm, at least 900 μm, at least 1 mm, at least 2 mm, at least 3 mm or at least 4 mm. This spacing may be at most 5 mm (corresponding to relatively low frequency SAWs), more preferably at most 4 mm, more preferably at most 3 mm, more preferably at most 2 mm. In some cases this spacing may be at most 1 mm, more preferably at most 0.9 mm, at most 0.8 mm, at most 0.7 mm, or at most 0.6 mm. For example, a cavity-to-cavity nearest neighbour spacing in the range 200-500 μm has been shown to be suitable for relatively small cavities. For higher frequencies, e.g. in the GHz range, smaller spacings are contemplated, e.g. in the range down to at least 1 μm. Spacing between the cavities is considered to be important in order to prevent liquid merging as it escapes from adjacent cavities.

The frequency of the acoustic wave may be in the range of about 10 kHz to about 1 GHZ, preferably about 1 MHz to about 100 MHz, more preferably about 5 MHz to about 50 MHz, more preferably about 5 MHz to about 20 MHz, more preferably about 15 MHz to about 5 MHz, more preferably between about 13 MHz and about 8 MHz. The frequency of the acoustic wave may be about 12 MHz, about 11 MHz, about 10 MHz, about 9 MHz or about 8 MHz.

The transducer may be formed from any suitable material for generating surface acoustic waves. Acoustics waves may be generated, for example, by a piezoelectric process, by a magnetostrictive process, by an electrostrictive process, by a ferroelectric process, by a pyroelectric process, by a heating process (e.g. using pulsed laser heating) or by an electromagnetic process. It is most preferred that the acoustic wave generation material layer is formed from a piezoelectric layer. In the disclosure set out below, the term “piezoelectric layer” is used but is it understood here that similar considerations would apply to acoustic wave generation material layers formed, for example, of magnetostrictive materials. Therefore, unless the context demands otherwise, the optional features set out in relation to the “piezoelectric layer” are to be understood as applying more generally to the acoustic wave generation material layer, when formed of any suitable material.

Preferably, the transducer comprises a “piezoelectric layer”, e.g. a sheet (e.g. a self-supporting sheet) of piezoelectric material. The layer of piezoelectric material may be a single crystal, such as a single crystal wafer. A suitable material is LiNbO3. A preferred orientation for the cut for this material is Y-cut rot. 128°. This has a higher electromechanical coupling coefficient than other orientations. Other ferroelectric materials may be used, e.g. PZT, BaTiO3, SbTiO3 or ZnO. Still further, materials such as SiO2 (quartz), AlN, LiTaO3, Al2O3 GaAs, SiC or polyvinylidene fluoride (PVDF) may be used. As an alternative to a single crystal, the material can be provided in polycrystalline or even amorphous form, e.g. in the form of a layer, plate or film.

Preferably, in use, when the acoustic wave interaction surface is facing upwards, the liquid is contained in the cavities such that the free surface of the liquid is below the level of the acoustic wave interaction surface. Thus, it is preferred that the free surface of the liquid is not located at or above the level of the acoustic wave interaction surface. This allows the liquid contained in the cavities to be isolated from each other, forbidding the formation of capillary waves at the liquid contained in the cavities.

The interior surface of the cavities may be treated in order to promote the containment of the liquid in the cavities. For aqueous liquids, preferably the interior surface of the cavities is formed to be hydrophilic. For non-aqueous liquids, preferably the interior surface of the cavities is formed to be hydrophobic.

Additionally or alternatively, the acoustic wave interaction surface may be treated in order to promote the containment of the liquid in the cavities. For example, this treatment may be selectively carried out at the array of cavities intended to contain the liquid. For aqueous liquids, preferably the acoustic wave interaction surface is formed to be hydrophilic. For non-aqueous liquids, preferably the acoustic wave interaction surface is formed to be hydrophobic. Preferably, an area of the acoustic wave interaction surface at which it is not intended for the liquid to be located is formed to be hydrophobic or hydrophilic, respectively, to promote the location of the liquid at the array of cavities intended to contain the liquid.

The respirable fraction of the droplets can be defined as the integral of the droplet size distribution in the diameter range 1-5 μm (N₁₋₅) divided by the integral of the droplet size distribution over the total diameter range measured (Nₜₒₜₐₗ). Thus, respirable fraction can be defined as (N₁₋₅)/(Nₜₒₜₐₗ).

Preferably, operation of the device results in a nebulised plume of droplets with a respirable fraction of at least 30%, more preferably at least 45%, more preferably at least 60%, more preferably about 80%, more preferably at least 90%, more preferably about 95% or higher.

In the prior art, it is known to filter out larger droplets from a nebulised plume in order to restrict the droplet size distribution which reaches the subject. However, this reduces the efficiency of the device, by reducing the proportion of the dose which reaches the subject, and clogging is also a problem, wherein captured large droplets prevent subsequent smaller droplets from being passed through. In the present invention, it is preferred that the respirable fraction is determined on the basis of the nebulised plume formed from the cavities, and not subjected to filtration prior to determination of the droplet size distribution.

Preferably, the device is capable of nebulising the liquid at a rate of at least 10 μl/min, more preferably at least 50 μl/min, more preferably at least 80 μl/min, more preferably at least 100 μl/min, more preferably at least 200 μl/min, more preferably at least 500 μl/min, more preferably at least 1 ml/min, more preferably at least 5 ml/min or higher.

The liquid may have relatively high viscosity, because the mechanism of the nebulisation provided in the present invention can tolerate relatively high viscosity. The viscosity of the liquid (measured at room temperature) may be at least 0.5 mPa·s, but in some embodiments may be at least 1 mPa·s, at least 5 mPa·s, or at least 10 mPa·s. For reference, at room temperature ethanol has viscosity of 1.07 mPa·s, bovine serum albumin 5% in phosphate buffer has viscosity of 1.5 mPa·s, glycerol has viscosity of 1200 mPa·s and water has viscosity of 0.894 mPa·s.

The surface tension of the liquid (measured at room temperature) may be at least 10 mN/m. In some embodiments, the surface tension may be at least 50 mN/m. For reference, at room temperature ethanol has a surface tension of 22.1 mN/m, bovine serum albumin 5% in phosphate buffer has a surface tension of 55.0 mN/m, glycerol has a surface tension of 63.0 mN/m and water has a surface tension of 71.9 mN/m.

The supply of liquid may be provided via a dispenser, for example, by a syringe pump or a piezo pump. Other metered liquid supply systems may be used. The dispenser may comprise a needle in fluid communication with a reservoir. An end of the needle may be spatially separated from the acoustic wave interaction surface, and not in contact with the transducer, such that the needle and the reservoir are isolated from the acoustic waves. Alternatively, the needle may be in contact with the acoustic wave interaction surface when dispensing the liquid. In some embodiments, the needle may be in fluid contact with the acoustic wave interaction surface during dispensing the liquid, i.e. there may be formed a liquid bridge between the needle and the acoustic wave interaction surface during dispensing the liquid.

In order to supply additional liquid to the cavities, it is possible for the cavities to be open at their end distal from the acoustic wave interaction surface. In that case, the distal ends of the cavities may be in fluid communication with a reservoir of the liquid, to be drawn up by capillarity into the cavities to replace liquid lost by nebulisation. In this case, it is possible for the liquid to be used as the coupling agent for the superstrate.

In order to provide adequate rate of nebulisation, the device may include a plurality of arrays of cavities, in order that there is a suitable number of cavities operating to contribute to the rate of nebulisation (in terms of the volume of liquid nebulised in total by the device per unit time). These may each be associated with a corresponding respective transducer. However, it is possible for the plurality of arrays of cavities to be operated using a single transducer. In order to provide a suitable distribution of acoustic waves to the respective arrays of cavities, the device may include phononic arrays, as set out in WO 2011023949, WO 2011060369, WO 2012114076 and/or WO 2012156755, in order to concentrate the acoustic wave distribution as required at the respective arrays of cavities.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 shows a schematic view of a system for the preparation and analysis of nebulised droplets according to a reference arrangement.

FIG. 2 shows a modified version of the system in FIG. 1, including a microcavity arrangement, according to an embodiment of the invention.

FIG. 3a and FIG. 3b show schematic enlarged views of parts of the system of FIG. 2.

FIG. 4 shows the APSD after nebulisation using the microstructure with 600 μm cavities at different SAW input powers using a flow rate of 60 μl/min. For each effective cut-off diameter on the horizontal axis, the left hand bar is for 4 W, the middle bar is for 6 W and the right hand bar is for 9 W.

FIG. 5 shows the APSD after nebulisation using the microstructure with 400 μm cavities at different SAW input powers using a flow rate of 60 μl/min. For each effective cut-off diameter on the horizontal axis, the left hand bar is for 4 W, the middle bar is for 6 W and the right hand bar is for 9 W.

FIG. 6 shows the mass median aerodynamic diameter (MMAD) at different SAW input powers for microstructures with 400 μm cavities and 600 μm cavities using a flow rate of 60 μl/min. For each input power on the horizontal axis, the left hand bar is for 400 μm cavities and the right hand bar is for 600 μm cavities.

FIG. 7 shows the MMAD at different input powers for a second microstructure with 600 μm cavities using a flow rate of 50 μl/min. For each input power on the horizontal axis, the left hand bar is for 2 point MMAD and the right hand bar is for full data MMAD.

FIG. 8 shows the fine particle fraction (FPF) at different input powers for the second microstructure with 600 μm cavities using a flow rate of 50 μl/min.

FIG. 9 shows the APSD after nebulisation using the second microstructure with 600 μm cavities at different SAW input powers using a flow rate of 50 μl/min. For each NGI stage on the horizontal axis, the left hand bar is for 4 W, the middle bar is for 6 W and the right hand bar is for 9 W.

FIG. 10 shows the mass median aerodynamic diameter (MMAD) generated using an embodiment of the invention at the same power but at different liquid flow rates.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

The present disclosure studies the nebulisation of liquids using surface acoustic waves (SAWs), although the use of different acoustic energy is also contemplated in order to achieve the advantages of the invention. This builds on the inventors' previous work (WO2016075209A1) the ability to control the aerodynamic particle size distribution (APSD) through use of different microstructures was demonstrated. In particular, the effect of increasing the power of the SAWs on the aerosol particle size distribution (APSD) is now considered. In a further study, the inventors' present work considers the effect of increasing the flow rate of the liquids to be nebulised on the (APSD).

It is demonstrated that the aerosol particle size diameter depends on the microstructure size and the input power. According to Nazarzadeh et al (2017), this phenomenon can be partially attributed to the microstructure's cavities, which physically confine the solution and restrict capillary wave excitation, thus controlling aerosol droplet size. Larger cavities result in lower capillary wave frequencies (and longer wavelengths) at the air-liquid interface, producing larger aerosol droplets. Moreover, the delivery of the solution into the microstructures is regulated by the acoustic wave propagation path and amplitude.

Embodiments of the invention are now described in view of experimental work carried out.

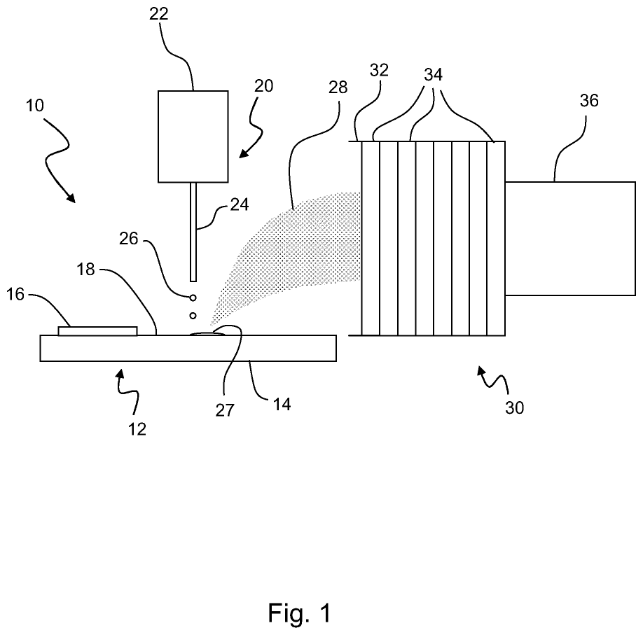

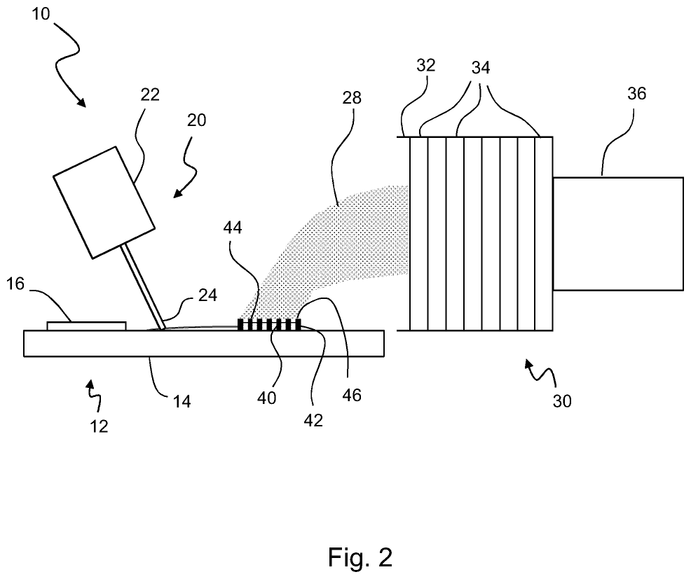

FIGS. 1 and 2 each show a system for the preparation of nebulised droplets. FIG. 1 is described first and the relevant differences for the system of FIG. 2 are then described.

In the system of FIG. 1, there is a nebuliser device 10 comprising an acoustic wave transducer 12 having a piezoelectric material substrate 14 and an electrode arrangement 16, typically in the form of an interdigitated electrode arrangement. The electrode arrangement 16 is located on the top surface of the substrate 14, which in this embodiment is planar. There is also a region of the top surface of the substrate 14 where the electrode arrangement is not located. This region includes an acoustic wave interaction surface 18 onto which a liquid is to be dispensed, as described later.

The system includes a dispenser 20 which comprises a reservoir 22 and a dispensing conduit 24, which in this example is a needle. The dispenser may also include a pump (not shown) in order to cause flow of and meter the liquid from the dispenser 20.

In operation, a suitable electrical drive signal is applied to the electrode arrangement 16. In combination with the piezoelectric substrate 14, this forms the transducer 12 that generates surface acoustic waves (SAWs) that propagate along the acoustic wave interaction surface 18. The SAWs interact with the liquid located on the acoustic wave interaction surface 18 to produce nebulised droplets of the liquid. The nebulised plume 28 is indicated schematically in FIG. 1.

In the arrangement shown in FIG. 1, the end of the dispensing needle 24 is spatially separated from the acoustic wave interaction surface 18, and not in contact with the transducer 12, such that the dispenser 22 is isolated from the SAWs propagating to and along the acoustic wave interaction surface 18. This configuration is particularly useful when the liquid is a suspension formulation since acoustic clustering of non-soluble particles in the formulation in the dispenser is avoided.

In some embodiments of the present invention, the drops 26 fall under gravity from the end of the dispensing needle 24 onto the acoustic wave interaction surface 18 at a predetermined flow rate. In other embodiments, the needle 24 may be in fluid contact with the acoustic wave interaction surface 18 during dispensing the liquid, i.e. there may be formed a liquid bridge between the needle 24 and the acoustic wave interaction surface 18 during dispensing the liquid.

The effect of the SAWs propagating along the surface results in some acoustic streaming of the liquid along the surface, as shown schematically in FIG. 1 by the sessile drop 27 being located slightly offset from the position at which drops 26 impact the acoustic wave interaction surface 18. It will be understood that different modes of delivery of the liquid to the acoustic wave interaction surface 18 are possible, for example by metering the isolated volumes of suspension formulation by spraying, slug flow or other equivalent approach.

In order to carry out the experiments reported here, the nebulised plume 28 is drawn in a particle size analyser 30. The analyser in the example of FIG. 1 uses an Andersen Cascade Impactor (ACI) with inlet 32, eight stages 34 of the cascade as described later and flow pump 36 to generate the air flow indicated in FIG. 1 as drawing the nebulised plume 28 towards and into the inlet 32. For the purpose of the experiments discussed herein, the ACI is interchangeable with a Next Generation Impactor (NGI) and it is expected that both types of analyser obtain comparable results.

FIG. 2 shows a similar overall arrangement to FIG. 1, identical features of which are not described again with respect to FIG. 2, but instead concentrating on the features and operation that make FIG. 2 an embodiment of the invention. The main difference with respect to FIG. 1 is that FIG. 2 has a microcavity arrangement 40 located on the surface of the substrate 14. This microcavity arrangement 40 is an arrangement of microcavities 44 formed through a plate 42 of material, the plate 42 of material being located on the upper surface of the substrate. In operation, the SAWs are coupled from the acoustic wave interaction surface 18 into the plate 42 (corresponding to the “superstrate” mentioned above). Additionally, the SAWs propagate to and along a second acoustic wave interaction surface 46 provided on an upper surface of the plate 42. The microcavities 44 are open at their end distal from the second acoustic wave interaction surface 46. This is known to improve the particle size distribution of the nebulised plume 28.

In the arrangement shown in FIG. 2, the needle 24 is configured in contact with the acoustic wave interaction surface 18 during dispensing the liquid. As such, the needle 24 dispenses the liquid directly onto the upper surface of the substrate 14 between the electrode arrangement 16 and the microcavity arrangement 40.

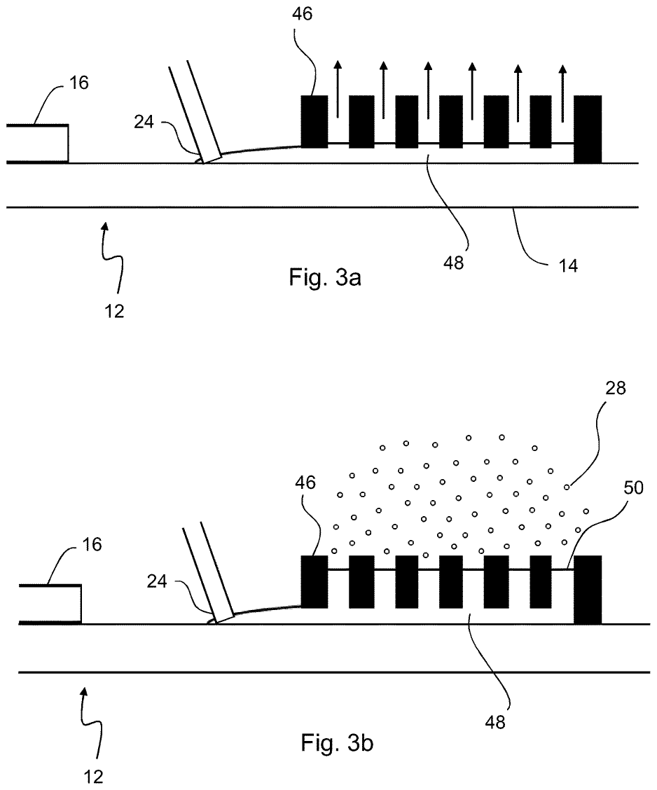

FIGS. 3a and 3b show enlarged views of part of the arrangement of FIG. 2 illustrating schematically the flow of the liquid from the dispensing needle 24. As shown in FIG. 3a, the microcavity arrangement 40 and the upper surface of the substrate 14 are configured to provide a gap therebetween. The drops 26 dispensed from the needle 24 collect on the upper surface of the substrate 14 to form a reservoir of liquid 48 in fluid communication with the gap. Accordingly, the distal ends of the cavities are in fluid communication with the reservoir 48. As depicted by the arrows in FIG. 3a, liquid from the reservoir 48 is drawn up by capillarity into the microcavities. FIG. 3b shows the liquid flow in a steady-state configuration wherein the supply of liquid to the reservoir 48 replaces liquid lost by nebulisation. In this case, the steady-state liquid level 50 within the microcavities lies below the second acoustic wave interaction surface 46. This is a preferred configuration of operation for the device since otherwise the liquid in the cavities would overtop the cavities and accumulate into one or more larger sessile drops on the upper surface of the superstrate.

Using a SAW nebuliser device and analyser setup based on the arrangement of FIG. 2, nebulisation experiments were conducted using an aqueous solution containing 1% sodium chloride (NaCl) and 1.32 mg/ml of Allura Red AC dye (ARAC). The aqueous solution was nebulised using different microstructures in each experiment, with cavity diameters D of 400 μm and 600 μm, respectively. The solution was delivered through a needle located near the microstructure and within the SAW propagation path, with acoustic wetting employed to introduce the solution into the respective microstructure. Specifically, a 32 G needle was used to deliver 400 μl of the aqueous solution placed in the propagation path of the surface acoustic waves. The process of wetting and aerosolisation was studied by manipulating the acoustic energy through adjusting the SAW input power at 4 W, 6 W and 9 W. The flow rate was kept constant at 60 ul/min.

The aerosol droplet size distribution was measured using an Andersen Cascade Impactor (ACI) (Copley Scientific Ltd, UK) at 28.3 L/min and a Next Generation Impactor (NGI) (Copley Scientific Ltd, UK) at 15 L/min, both operating at 4° C. to reduce evaporation effects. Both impactors were used in order to increase speed of collecting data. The aerosol droplet size distribution was determined by measuring the ARAC concentration at each cascade stage using a UV-Vis Spectrophotometer (VWR International LLC, UK). Standard curves for ARAC quantification were created prior to nebulisation. All experiments were carried out in triplicate and average values with standard deviation are reported.

The mass median aerodynamic diameter (MMAD) was found by interpolation of the two points on the cumulative mass distribution graph that surround the 50% cumulative value or D50 (Christopher et al, 2010). The fine particle fraction (FPF) was calculated as the percentage cumulative fraction of the ARAC mass in the aerosol relevant to each equipment, i.e., below 5.39 μm in size for the NGI and below 5.8 μm in size for the ACI. Additionally, a two-tailed t-test was performed on the obtained MMADs and the results were considered statistically significant if the p-value was less than 0.05.

Table 1 displays the results of aerosol characterization from the NGI using the MMAD and FPF produced with different SAW input powers.

TABLE 1
MMAD and FPF of aerosols generated at varying SAW input
powers using microstructure with 600 μm cavities.

| SAW Input power | MMAD (μm) | FPF (%) |
|---|---|---|
| 4 W | 1.9 ± 0.1 | 57.0 ± 5.5 |
| 6 W | 3.2 ± 0.1 | 30.6 ± 2.9 |
| 9 W | 4.7 ± 0.4 | 22.8 ± 3.0 |

The results reveal an increase in MMAD and a corresponding decrease in FPF as power increases. The MMAD is seen to increase from 1.9 μm to 4.7 μm as SAW input power increases from 4 W to 9 W. As the MMAD increases to values around 5 μm, it becomes evident that 50% of the aerosols are larger than a 5 μm cut-off. Consequently, the fraction of aerosols smaller than 5 μm (FPF) decreases, demonstrating a shift towards larger aerosol sizes. This is an unexpected result, since previous consideration of the mechanisms taking place in the cavities may have suggested that increasing the power would lead to the formation of smaller nebulised droplets.

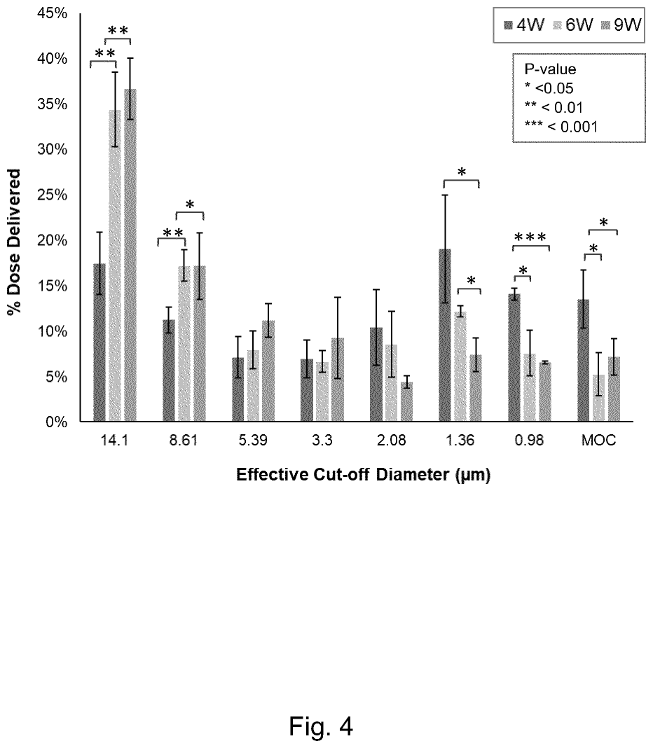

FIG. 4 presents the mass distribution of ARAC corresponding to the percentage of dose delivered across different stages of the NGI represented by the effective cut-off diameter of each stage after nebulisation for each SAW input power using the microstructure with 600 μm cavities. The error bars represent the standard deviation of values.

The results show the bimodal distribution expected for nebulisation. A clear trend of APSD shifting towards larger aerosol sizes is observed with an increase in the SAW input power which can be associated with acoustic energy.

Table 2 shows the MMAD and FPF of the aerosolised droplets collected from the ACI at various SAW input powers for a microstructure with 400 μm cavities. The MMAD is seen to increase from 0.7 μm to 1.1 μm as SAW input power increases from 4 W to 9 W. Meanwhile, the FPF decreases as SAW input power increases from 6 W to 9 W. This trend aligns with the observations made using the 600 μm microstructure, providing additional evidence of the shift towards larger aerosol particle sizes.

TABLE 2
MMAD and FPF of aerosols generated at varying SAW input
powers using microstructure with 400 μm cavities.

| SAW Input power | MMAD (μm) | FPF (%) |
|---|---|---|
| 4 W | 0.7 ± 0.1 | 83.2 ± 2.2 |
| 6 W | 0.8 ± 0.1 | 83.8 ± 2.2 |
| 9 W | 1.1 ± 0.1 | 54.4 ± 5.9 |

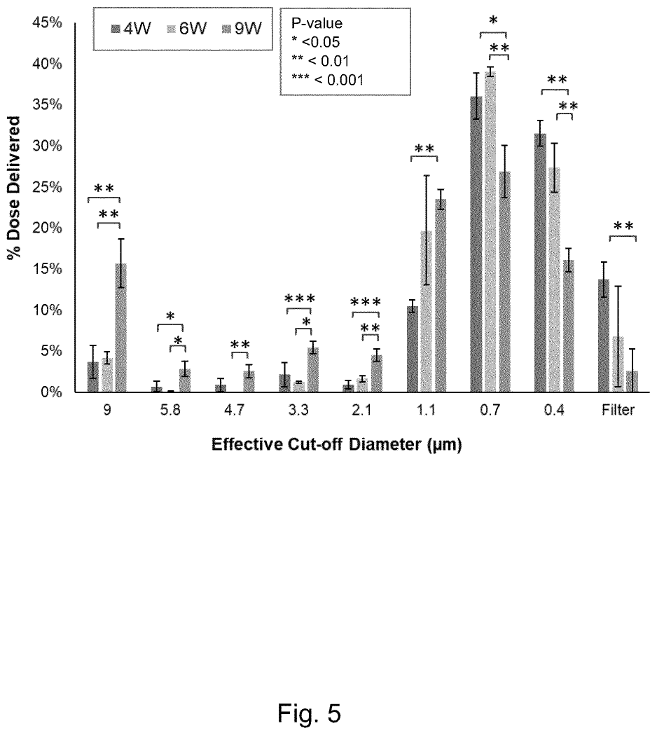

FIG. 5 shows the mass distribution of ARAC corresponding to the percentage of dose delivered across different stages of the ACI for each SAW input power using the microstructure with 400 μm cavities. The error bars represent the standard deviation of values. A trend of APSD shifting towards larger aerosol sizes is observed with an increase in the SAW input power which can be associated with acoustic energy.

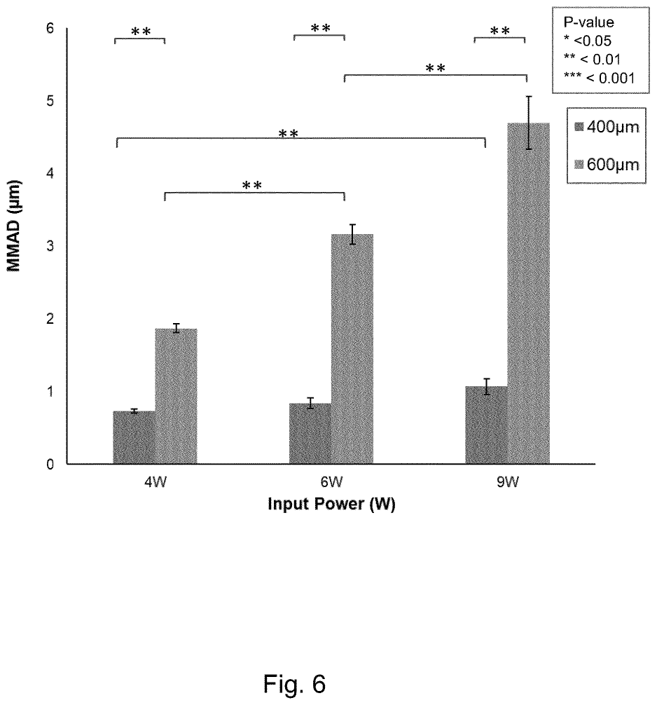

FIG. 6 shows the MMAD calculated at different SAW input powers for each microstructure, with 400 μm cavities and 600 μm cavities, respectively. As shown in FIG. 6, two-tailed t-test analyses were performed to compare the MMAD results between the 400 μm and 600 μm cavities at SAW power levels of 4 W, 6 W, and 9 W. The results were statistically significant as defined by p-values of less than 0.05, indicating a meaningful difference in the MMAD between the two sets of cavities at the different SAW input powers. The error bars correspond to the standard deviation.

A further study similarly investigating the effect of increasing the power of the SAWs on the aerosol particle size distribution (APSD) is discussed below.

Using a SAW nebuliser device with a different microstructure comprising 600 μm cavities with indent and an NGI analyser setup based on the arrangement of FIG. 2, nebulisation experiments were conducted using an aqueous solution containing 1% sodium chloride (NaCl) and an analyte dye. The solution was delivered through a 32 G needle located near the microstructure on the side of the transducer and within the SAW propagation path, with acoustic wetting employed to introduce the solution into the respective microstructure. A syringe pump was used to regulate the amount of liquid supplied to the superstrate continuously at a constant flow rate of 50 ul/min using a pump pressure of 25 mBar. The process of wetting and aerosolisation was studied by manipulating the acoustic energy through adjusting the SAW input power at 4 W (−8.5 dBm), 6 W (−7 dBm), 9 W (−5 dBm).

The aerosol droplet size distribution was measured using a Next Generation Impactor (NGI) (Copley Scientific Ltd, UK) at 15 L/min, operating at 4° C. to reduce evaporation effects. The aerosol droplet size distribution was determined by measuring the analyte concentration at each cascade stage using a UV-Vis Spectrophotometer (VWR International LLC, UK). Standard curves for ARAC quantification were created prior to nebulisation. All experiments were carried out in triplicate (n=3) and average values with standard deviation are reported.

The two-point mass median aerodynamic diameter (‘2 point MMAD’) was found by interpolation of the two points on the cumulative mass distribution graph that surround the 50% cumulative value or D50 (Christopher et al, 2010), while the ‘Full data MMAD’ was obtained using the full spectrum of aerosol particles. The fine particle fraction (FPF) was calculated as the percentage cumulative fraction of the analyte mass in the aerosol below 5 μm in size.

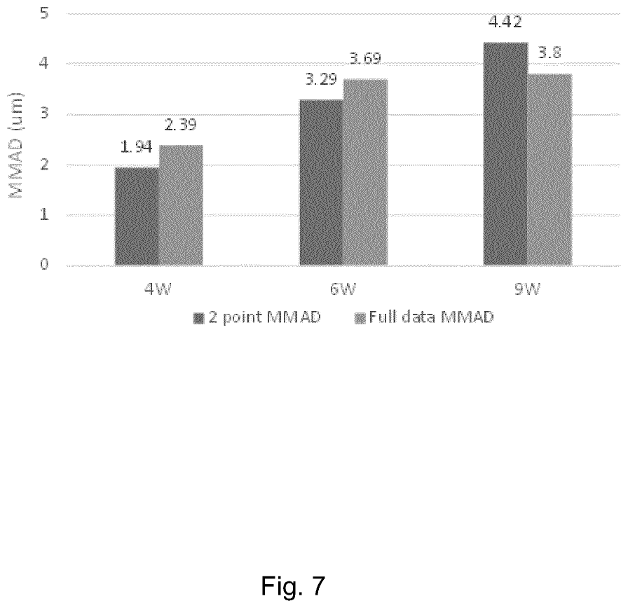

FIG. 7 shows the MMAD of the nebulised droplets as a function of input power. The two-point MMAD increased from 1.94 μm to 4.42 μm as the input power increased from 4 W to 9 W. Therefore, the average particle size increases with input power confirming the trend established in the previous study (see Tables 1 and 2).

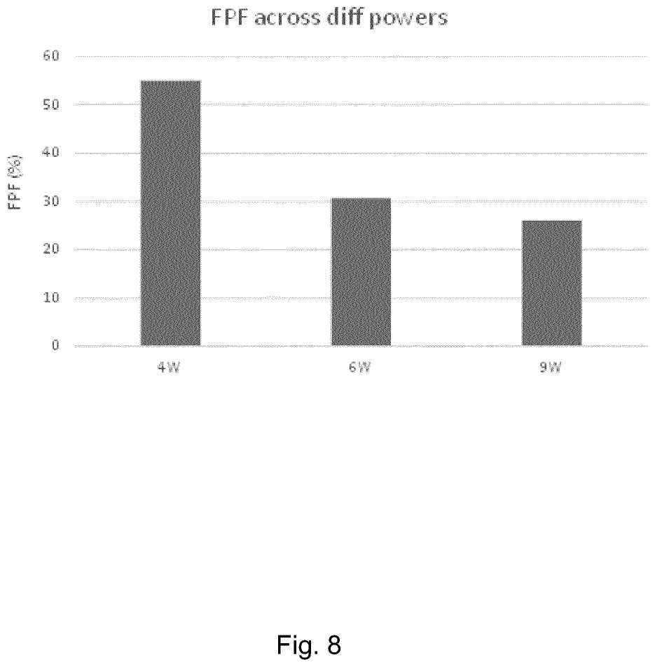

FIG. 8 shows FPF of the nebulised droplets as a function of input power. The FPF is shown to decrease as the input power was increased from 4 W to 9 W. This corroborates the trend established in the previous study (see Tables 1 and 2).

Particle size ranges of the nebulised droplets collected in each cascade stage of the NGI, respectively, are indicated in Table 3.

TABLE 3
Particle cut off diameter for individual
stages in Next Generation Impactor

| | |
|---|---|
| Aerosol particle cut off diameter | Stage |

| | | |
|---|---|---|
| >14.1 | μm | 1 |
| 14.1-8.61 | μm | 2 |
| 8.61-5.39 | μm | 3 |
| 5.39-3.30 | μm | 4 |
| 3.30-2.08 | μm | 5 |
| 2.08-1.36 | μm | 6 |
| 1.36-0.98 | μm | 7 |
| <0.98 | μm | MOC |

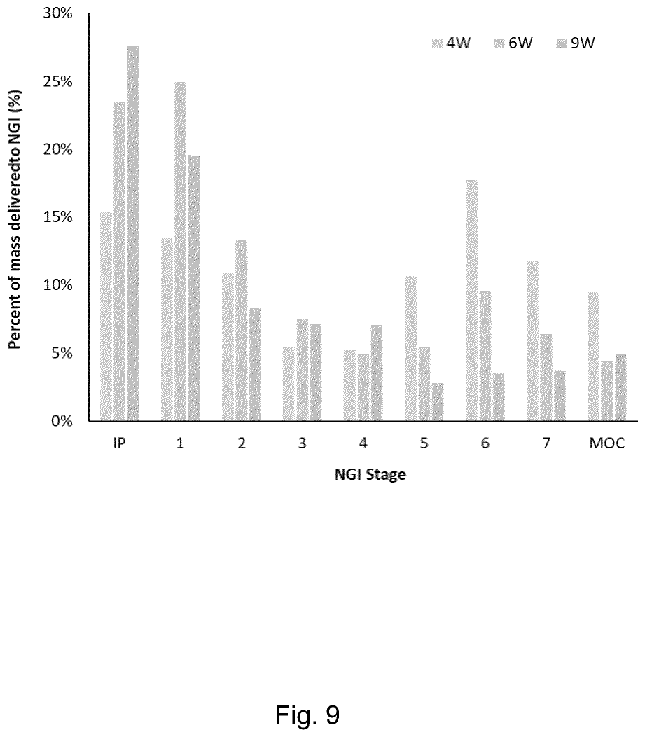

FIG. 9 shows the mass distribution of the analyte delivered to the NGI across the different stages of the NGI for each input power. A trend of APSD shifting towards larger aerosol sizes with an increase in the SAW input power is observed. In particular, a higher mass percentage was measured in the latter stages (4 to 7), corresponding to smaller particle sizes, for an input power of 4 W than an input power of 9 W.

In yet another study, the present inventors investigated the effect of increasing the flow rate of the aqueous solution on the APSD. The cavities were formed to extend through the superstrate allowing the cavities to be replenished with liquid by filling from below under capillarity. A pump was employed to regulate the amount of liquid supplied to the superstrate continuously. The aqueous solution was nebulised using the microstructure with 600 μm cavities and an input power of 9 W. The pump was set to an initial flow rate of 70 μL/min to maintain continuous production or rapid generation of nebulised droplets. The experiment was repeated for increased flow rates of 170 μL/min and 250 μL/min.

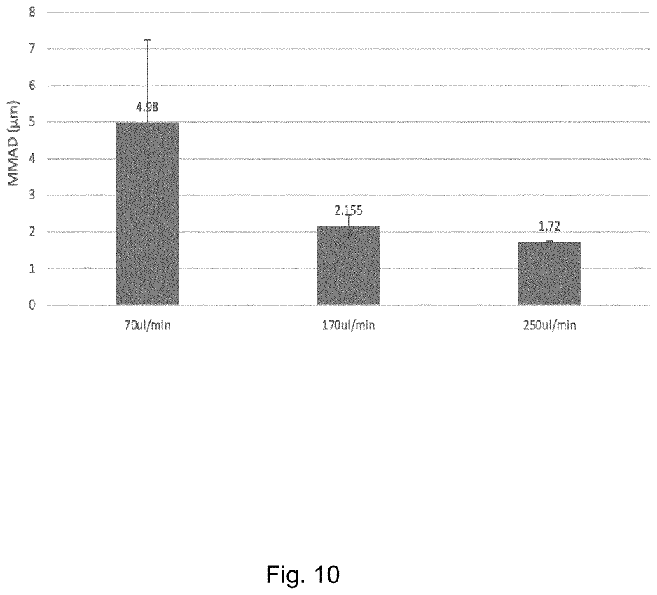

FIG. 10 shows the MMAD calculated for the different flow rates. The error bars represent the standard deviation of values. The MMAD was found to decrease from 4.98 μm to 1.72 μm as the flow rate increased from 70 μL/min to 250 μL/min. As such, increasing the flow rate has the opposite effect on the average aerosol particle size to that of increasing input power. This is again unexpected. Without wishing to be limited by theory, the present inventors believe that this effect can be attributed to the steady-state liquid level 50 in the microcavities 44. More particularly, the microcavities 44 can be partially-filled by the liquid (as shown in FIG. 3b) such that a microcavity 44 containing a lower volume of liquid will have a high input power to liquid volume ratio. In other words, the ratio of input power to liquid volume increases as the liquid level 50 increases. As this ratio increases, rather than controlled nebulisation at the surface of the liquid in the microcavities 44, the liquid “explodes”. It is believed that this is caused by excitation of lower capillary frequencies (with longer capillary wavelength) by the acoustic waves.

In light of this, the average particle size can be controlled by varying the input power and/or varying the flow rate. Specifically, the average particle size may be increased by one or more of increasing the input power and decreasing the flow rate. Conversely, the average particle size may be reduced by one or more of decreasing the input power and increasing the flow rate. Accordingly, the inventors have found that the variation in droplet size may be managed by the ratio between the liquid flow rate and the input power. As such, the flow rate may be increased to facilitate the use of a higher input power, while controlling the average size of the nebulised droplets.

The median average aerosol droplet size of the aerosols measured in the experiments described above range from about 0.7 μm to about 5 μm. As discussed above, this range is particularly suited pulmonary drug delivery. Therefore, the present invention provides a single platform for targeted delivery to different regions of the lung.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### REFERENCES

A number of publications are cited above in order to more fully describe and disclose the invention and the state of the art to which the invention pertains. Full citations for these references are provided below. The entirety of each of these references is incorporated herein.
P. P. H. L. Brun, A. H. de Boer, H. W. Frijlink, and H. G. M. Heijerman, Pharm. World Sci., 2000, 22, 75-81.
Nazarzadeh, E. et al. ‘Confinement of surface waves at the air-water interface to control aerosol size and dispersity,’ Physics of Fluids 2017, 29 (11), p.112105.
Nazarzadeh, E. et al. ‘Controlling the size of nebulised droplets by pinning surface waves for precise delivery of aerosolised medicine,’ Journal of Aerosol Medicine and Pulmonary Drug Delivery 2019, 32 (2), p. A22-A23.
Pritchard, J. N. et al. ‘Next Generation Nebulized Therapy: Opportunities for New Treatments and Devices,’ Respiratory Drug Delivery 2022, 1 (2022), p. 103-112
Collins, D. J. et al. ‘Atomization off thin water films generated by high-frequency substrate wave vibrations,’ Physical Review E 2012, 86, 056312.
Christopher, J. D. et al. ‘Generalized simplified approaches for mass median aerodynamic diameter determination,’ Pharmacopeial Forum 2010, 36 (3).
J. Blamey, L. Y. Yeo, and J. R. Friend, Microscale Capillary Wave Turbulence Excited by High Frequency Vibration, Langmuir 2013, 29, 3835-3845.

1. A method for the preparation of nebulised droplets, the method including the steps:
providing a device having an acoustic wave interaction surface with an array of cavities opening to the acoustic wave interaction surface;
providing an acoustic wave transducer adapted to generate and propagate acoustic waves to the acoustic wave interaction surface;
providing, in the cavities, a liquid for nebulisation;
operating the acoustic wave transducer at a first input power to generate and propagate acoustic waves to the acoustic wave interaction surface to interact with the liquid in the cavities to produce nebulised droplets having a first aerosol particle size distribution; and
operating the acoustic wave transducer at a second input power, greater than the first input power, to generate and propagate acoustic waves to the acoustic wave interaction surface to interact with the liquid in the cavities to produce nebulised droplets having a second aerosol particle size distribution having a larger mass median aerodynamic diameter (MMAD) than that of the first aerosol particle size distribution.

2. The method according to claim 1, including adjusting the input power to the acoustic wave transducer from the first input power to the second input power and/or adjusting the input power of the acoustic wave transducer from the second input power to the first input power.

3. The method according to claim 1, wherein the input power to the acoustic wave transducer is variable through a range not less than 100 mW and not more than 30 W.

4. The method according to claim 1, including a power calibration step of calibrating the input power based on the first or second aerosol particle size distributions.

5. The method according to claim 4, wherein the power calibration step includes determining a desired average particle size, calculating an average particle size from the first or second aerosol particle size distributions, comparing the average particle size to the desired average particle size, and increasing or decreasing the input power based on the comparison.

6. The method according to claim 1, further including the steps:
metering the liquid for nebulisation to the cavities at a flow rate; and
altering the flow rate of liquid for nebulisation to the cavities, thereby altering the aerosol particle size distribution of the nebulised droplets.

7. The method according to claim 6, including increasing the input power and the flow rate simultaneously while maintaining a constant ratio of the input power to the flow rate of the liquid.

8. A method for the preparation of nebulised droplets, the method including the steps:
providing a device having an acoustic wave interaction surface with an array of cavities opening to the acoustic wave interaction surface;
providing an acoustic wave transducer adapted to generate and propagate acoustic waves to the acoustic wave interaction surface;
providing, in the cavities, a liquid for nebulisation by metering the liquid for nebulisation to the cavities at a flow rate;
operating the acoustic wave transducer to generate and propagate acoustic waves to the acoustic wave interaction surface to interact with the liquid in the cavities to produce nebulised droplets having an aerosol particle size distribution; and
altering the flow rate of liquid for nebulisation to the cavities and thereby altering the aerosol particle size distribution of the nebulised droplets.

9. The method according to claim 8, wherein altering the flow rate includes:
operating the device using a first flow rate to produce nebulised droplets having a third aerosol particle size distribution; and
operating the device using a second flow rate, smaller than the first flow rate, to produce nebulised droplets having a fourth aerosol particle size distribution having a larger mass median aerodynamic diameter (MMAD) than that of the third aerosol particle size distribution.

10. The method according to claim 9, including altering the flow rate from the first flow rate to the second flow rate and/or altering the flow rate from the second flow rate to the first flow rate.

11. The method according to claim 9, including a flow rate calibration step of calibrating the flow rate based on the third or fourth aerosol particle size distributions.

12. The method according to claim 11, wherein the flow rate calibration step includes determining a desired average particle size, calculating an average particle size from the third or fourth aerosol particle size distributions, comparing the average particle size to the desired average particle size, and increasing or decreasing the flow rate based on the comparison.

13. The method according to claim 1, wherein a maximum dimension of the cavities in a direction perpendicular to the depth of the cavities is not less than 200 μm.

14. The method according to claim 1, wherein the depth of the cavities is at least 500 nm.

15. The method according to claim 1, wherein each cavity is open at an end opposite to the acoustic wave interaction surface in fluid communication with a reservoir of the liquid to be drawn up by capillarity into the cavities to replace liquid lost by nebulisation.

16. The method according to claim 1, wherein operation of the device results in a nebulised plume of droplets of average diameter in the range 1 to 10 μm.

17. The method according to claim 1, wherein the liquid comprises one or more of: a pure compound; a mixture of liquids; a solution of one or more solutes in a liquid solvent; a suspension of particles (solid, substantially solid or liquid) in a carrier liquid; a colloid; an emulsion; nanoparticles or a suspension of nanoparticles.

18. The method according to claim 1, wherein the liquid comprises an active pharmaceutical ingredient.
